(19) United States
(12) Patent Application Publication
(10) Pub. No.: US 2010/0282980 A1
(43) Pub. Date: Nov. 11, 2010

(54) Stable Calibration Means for Apparatus for Photo Reduction of Contaminants in Blood

(75) Inventors: Abraham BELKIND, North Plainfield, NJ (US); Daniel Thomas MCGINNIS, Lakewood, CO (US); Dennis J. HLAVINKA, Arvada, CO (US); Terrence M. CUSSEN, Englewood, CO (US)

Correspondence Address: CaridianBCT, Inc.;Mail Stop: 810 1F2, 10811 WEST COLLINS AVE, LAKEWOOD, CO, 80215, US

(73) Assignee: CARIDIANBCT BIOTECHNOLOGIES, LLC, Lakewood, CO (US)

(21) Appl. No.: 12/760,627

(22) Filed: Apr. 15, 2010

Related U.S. Application Data
(60) Provisional application No. 61/177,124, filed on May 11, 2009.

(51) Int. Cl. A61L 2/08; H01L 31/0232; G02B 5/10; C23C 16/513

(52) U.S. Cl. 250/432 R; 250/216; 359/883; 427/534

(57) ABSTRACT

An apparatus for irradiating blood or blood products, preferably with ultra violet or visible light, to reduce contaminants in the blood or blood products. A removable radiometer having light integrating chambers detects the light intensity, allowing the radiation characteristics of the apparatus to be calibrated. The light chambers have an aluminum reflecting surface prepared by machining, grit blasting, polishing and plasma treatment including plasma cleaning and oxidation.

### CROSS REFERENCE TO RELATED APPLICATIONS

This application claims the benefit of U.S. Provisional Patent Application No. 61/177,124, filed on May 11, 2009, which is expressly incorporated herein by reference.

This application is related to U.S. application Ser. No. 12/032,963, which describes an apparatus for irradiating blood or blood products, preferably with ultra violet or visible light, to reduce contaminants in the blood or blood products. A removable radiometer having light integrating chambers detects the light intensity, allowing the radiation characteristics of the apparatus to be calibrated. This application describes an improvement to the radiometer comprising a surface for a light integrating chamber with stable optical characteristics. The radiometer with such a surface can provide accurate calibration for the blood-irradiating apparatus over an extended period of time.

### BACKGROUND

Contamination of whole blood or blood products with infectious microorganisms such as HIV, hepatitis and other viruses and bacteria present a serious health hazard for those who must receive transfusions of whole blood or administration of various blood products or blood components such as platelets, red cells, blood plasma, Factor VIII, plasminogen, fibronectin, anti-thrombin III, cryoprecipitate, human plasma protein fraction, albumin, immune serum globulin, prothrombin complex plasma growth hormones, and other components isolated from blood. Blood screening procedures may miss pathogenic contaminants, and sterilization procedures which do not damage cellular blood components but effectively inactivate all infectious viruses and other microorganisms have not heretofore been available.

In some circumstances, certain blood components may themselves be harmful to the desired blood product. For example, white blood cells, which are part of the donor's immune system, may cause an adverse reaction in the recipient of a red blood cell product. Many white cells are separated by centrifugation from the desired red blood cells, but some usually remain mixed with the red blood cells. The undesired white blood cells may be considered a “contaminant” or “pathogen” with respect to the desired relatively pure red blood cell product. The white blood cells may be inactivated in the same manner as an infectious virus or microorganism.

The use of pathogen inactivating agents include certain photo sensitizers, or compounds which absorb light of defined wavelengths and transfer the absorbed energy to an energy acceptor, have been proposed for inactivation of microorganisms found in blood products or fluids containing blood products. Such photo sensitizers may be added to the fluid containing blood or blood products and irradiated.

The photo sensitizers which may be used in this invention include any photo sensitizers known to the art to be useful for inactivating microorganisms. A “photo sensitizer” is defined as any compound which absorbs radiation at one or more defined wavelengths and subsequently utilizes the absorbed energy to carry out a chemical process. Examples of photo sensitizers which may be used for the reduction of pathogens in blood or blood products include porphyrins, psoralens, dyes such as neutral red, methylene blue, acridine, toluidines, flavine (acriflavine hydrochloride) and phenothiazine derivatives, coumarins, quinolones, quinones, and anthroquinones.

A number of systems and methods for irradiating pathogens in a fluid with light either with or without the addition of a photo sensitizer are known in the art. Examples include U.S. Pat. No. 5,762,867; U.S. Pat. No. 5,527,704; U.S. Pat. No. 5,868,695; U.S. Pat. No. 5,658,722; and U.S. Pat. No. 6,843,961.

Use of light which is variably pulsed at a wavelength of 308 nm without the addition of a photo sensitizer to inactivate virus in a washed platelet product is taught in an article by Prodouz et al. (Use of Laser-UV for Inactivation of Virus in Blood Products; Kristina Prodouz, Joseph Fratantoni, Elizabeth Boone and Robert Bonner; Blood, Vol 70, No. 2).

Whether or not a photo sensitizer is used, it is important that the dosage of radiation delivered to the blood or blood component be accurately controlled. Proper calibration of the irradiation apparatus is, therefore, necessary.

### SUMMARY OF THE INVENTION

The present invention provides an apparatus for irradiating a fluid containing blood products and pathogens, including a radiometer having a stable optical surface in a light-integrating cavity for accurate calibration of delivered radiation. The apparatus comprises a treatment chamber having at least one radiation emitting source; a support platform for holding the fluid containing blood cells or blood components to be irradiated; a control unit for controlling the radiation emitting source; and a removable radiometer in electrical communication with the control unit, the radiometer comprising a first optical chamber having a aperture for receiving at least some of the radiation and a photo sensor responsive to the received radiation in the optical chamber. The optical chamber may comprise an elongated cavity or cylinder, with an aperture shaped as a slot extending parallel to a long dimension of the optical chamber. This aperture might be covered by or filled with a light transmitting material such as quartz glass. An inner surface of the optical chamber may be “optically rough”, producing a diffuse or lambertion reflection. The inner surface according to this invention comprises an aluminum substrate treated by bead blasting and surface polishing. The inner surface may be further coated with a layer of aluminum oxide, substantially comprising artificial sapphire. Preferably, the aluminum oxide layer is between about 200 nm and 600 nm in thickness. A 200-400 nm range appears to have produced the best stability for flat coupons. For cavities, the best stability was around 400-600 nm.

The preferred optical surface may be produced by providing an aluminum substrate. Preferably, the substrate comprises 1100 aluminum. The substrate is bead blasted to produce an optically rough surface, and surface polished to remove excessive irregularities.

The polished surface of aluminum is then cleaned chemically (by using ultrasonic energy to produce agitation and cavitation at the optical surface with de-ionized water) and by plasma cleaning by placing the surface in a plasma treatment chamber.

In a further aspect, a layer of aluminum oxide may be produced on the substrate by keeping the substrate in the plasma treatment chamber. Oxygen atoms and ions created in a plasma within the chamber react with an exposed surface forming aluminum oxide. Preferably a layer of aluminum oxide is produced having a thickness of between about 200 nm and 600 nm.

These and other features of the invention will be apparent from the following detailed description, taken with reference to the accompanying drawings.

### BRIEF DESCRIPTION OF THE DRAWINGS

FIG. 1 is a cross-sectional view of a treatment chamber in an illuminator which may be used in the present invention.

FIG. 2 is a perspective view of an irradiation apparatus or illuminator containing a treatment chamber.

FIG. 3 is a cross-sectional plan view of a radiometer, taken along line 3-3 of FIG. 6 positioned on a platen in the illuminator of FIG. 2.

FIG. 4 is a perspective view of a radiometer.

FIG. 5 is a further perspective view of the radiometer of FIG. 4.

FIG. 6 is a perspective view of an assembled radiometer.

FIG. 7 is a perspective view of an apparatus for plasma treatment.

FIG. 8 is a perspective view of a cradle for holding parts in the apparatus of FIG. 7.

FIG. 9 is a plane view of the apparatus of FIG. 7.

FIG. 10 is a graph of photodiode signal change.

FIG. 11 is a graph of change of the ratio of efficiency to stability in the cavity of the radiometer.

FIG. 12 is a graph of the photo diode signal from 250 nm to 400 nm.

### DETAILED DESCRIPTION

The term “blood product” as used herein includes all blood constituents or blood components and therapeutic protein compositions containing proteins derived from blood as described above. Fluids containing biologically active proteins other than those derived from blood may also be treated by the methods and devices of this invention.

Photo sensitizers may include compounds which preferentially adsorb to nucleic acids, thus focusing their photodynamic effect upon microorganisms and viruses with little or no effect upon accompanying cells or proteins. Other types of photo sensitizers are also useful in this invention, such as those using singlet oxygen-dependent mechanisms.

Most preferred are endogenous photo sensitizers. The term “endogenous” means naturally found in a human or mammalian body, either as a result of synthesis by the body or by ingestion as an essential foodstuff (e.g. vitamins) or by formation of metabolites and/or byproducts in vivo. Examples of such endogenous photo sensitizers are alloxazines such as riboflavin, lumiflavin, lumichrome, flavine adenine dinucleotide, alloxazine mononucleotide, vitamin Ks, vitamin L, and other compounds. When endogenous photo sensitizers are used, no removal or purification step is required after decontamination, and a treated product can be directly returned to a patient's body or administered to a patient in need of its therapeutic effect without any further required processing. Using endogenous photo sensitizers to inactivate pathogens in a blood product are described in U.S. Pat. No. 6,843,961, No. 6,258,577 and No. 6,277,337, herein incorporated by reference in their entirety to the extent consistent with this disclosure.

The fluid to be pathogen inactivated has the photo sensitizer added thereto, and the resulting fluid mixture may be exposed to photo radiation of the appropriate peak wavelength and amount to activate the photo sensitizer, but less than that which would cause significant non-specific damage to the biological components or substantially interfere with biological activity of other proteins present in the fluid. Accurate control of the amount of radiation delivered to the fluid is, therefore, important.

The term peak wavelength as defined herein means that the light is emitted in a narrow range centered on a wavelength having particular peak intensity. Visible light for pathogen reduction may be centered on a wavelength of approximately 470 nm, and having a maximum intensity at approximately 470 nm. In another embodiment, the light may be centered on a narrow range of UV light at an approximate wavelength of 302 nm, and having a maximum intensity at approximately 302 nm. The term light source or radiation source as defined herein means an emitter of radiant energy, and may include energy in the visible and/or ultraviolet range, as further described below.

The fluid containing the photo sensitizer may also be flowed into and through a photo-permeable container for irradiation, using a flow through type system. Alternatively, the fluid to be treated may be placed in a photo-permeable container which is agitated and exposed to photo radiation for a time sufficient to substantially inactivate the microorganisms, in a batch-wise type system.

The term “container” refers to a closed or open space, which may be made of rigid or flexible material, e.g., may be a bag or box or trough. The container may be closed or open at the top and may have openings at both ends, e.g., may be a tube or tubing, to allow for flow-through of fluid therein. A cuvette has been used to exemplify one embodiment of the invention involving a flow-through system. Collection bags, such as those used with the Trima® and/or Spectra™ apheresis systems of CaridianBCT, Inc., Lakewood, Colo., USA, have been used to exemplify another embodiment involving a batch-wise treatment of the fluid.

The term “photo-permeable” means the material of the treatment container is adequately transparent to photo radiation of the proper wavelength for activating the photo sensitizer. After treatment, the blood or blood product may be stored for later delivery to a patient, concentrated, infused directly into a patient or otherwise processed for its ultimate use.

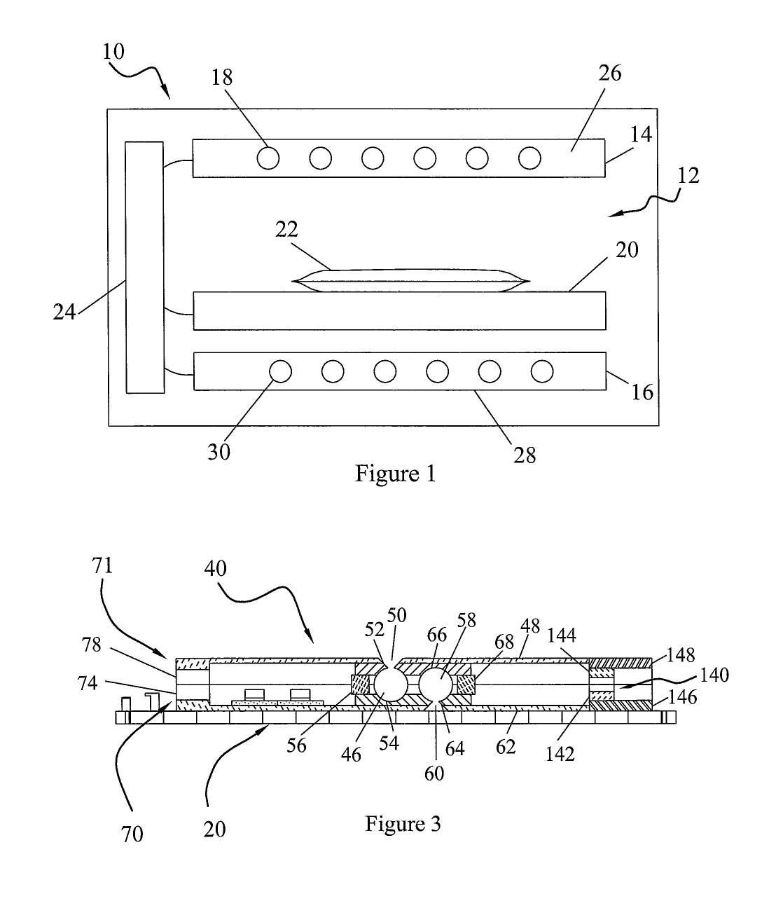

FIG. 1 shows, in a cross-sectional view, the inside of a radiation or treatment chamber of one type of apparatus that may be used in the present invention. The treatment chamber shown in FIG. 1 may be used in batch-wise systems; however, similar elements may also be used in flow-through systems. The apparatus 10, used for inactivating a fluid which may contain pathogens, consists of a radiation chamber 12 having at least one source of radiation 14. In one preferred embodiment (FIG. 1), the radiation chamber may contain a second source of radiation 16. A single light source may also be used. Each radiation source 14 and 16 respectively, is depicted as including a plurality of discrete radiation-emitting elements 18, 30. The radiation chamber 12 further consists of a support platform 20 for supporting a fluid container 22 containing the fluid to be irradiated, and a control unit 24.

As introduced above, two sources of radiation are shown within radiation chamber 12. Radiation source 14 may be located along the top portion of the radiation chamber 12 above the container 22, which holds or contains the fluid to be irradiated, while radiation source 16 may be located along the bottom portion of the radiation chamber 12 below the container 22. Although not shown, radiation sources may also be located along some or all of the sides of the radiation chamber 12 perpendicular to the container 22.

The upper radiation source 14 includes an upper support substrate 26 supporting a plurality of discrete radiation emitting elements or discrete light sources (see discrete source 18 as one example) mounted thereon. As further depicted in FIG. 1, the lower radiation source 16 includes a lower support substrate 28 which also supports a plurality of discrete radiation emitting elements or discrete light sources (see discrete source 30 as another example). Lower support substrate 28 preferably runs parallel to support platform 20. The support substrates 26, 28 may be substantially flat as shown, or may be in an arcuate shape, or may be in a shape other than arcuate, without departing from the spirit and scope of the invention.

The support substrate may or may not have reflective surfaces. In a further alternative configuration, the reflective surface may not contain any light sources. Such a reflective surface containing no light sources (not shown) may be located within the radiation chamber 12 on a side opposite from the radiation source. The support platform 20 may have a reflective surface 32. This reflective surface 32 on support platform 20 may be in place of, or may be in addition to another reflective surface within the radiation chamber. There may also be no reflective surfaces at all within the radiation chamber.

In any of these reflective surface embodiments, the reflective surface may be coated with a highly reflective material which serves to reflect the radiation emitted from the lights back and forth throughout the treatment chamber until the radiation is preferably completely absorbed by the fluid being irradiated. The highly reflective nature of the reflective surface reflects the emitted light back at the fluid-filled bag or container 22 with minimum reduction in the light intensity.

In FIG. 1, support platform 20 is positioned within the radiation chamber 12. The support platform 20 may be located substantially in the center of the radiation chamber (as shown in FIG. 1), or may be located closer to either the top portion or the bottom portion of the treatment chamber. The support platform 20 supports the container 22 containing the fluid to be irradiated. Additionally or alternatively, the platform 20 may be made of a photo-permeable material to enable radiation emitted by the lights to be transmitted through the platform and penetrate the fluid contained within the container 22. The platform may also be a wire or other similar mesh-like material to allow maximum light transmissivity therethrough.

The support platform 20 is preferably capable of movement in multiple directions within the radiation chamber 12. One type of agitation system used might be similar to the Helmer flatbed agitation system available from Helmer Corp. (Noblesville, Ind., USA). This type of agitator provides to and fro motion. Other types of agitators may also be used to provide a range of motion to the fluid contained within the container 22. For example, the support platform might be oriented in a vertical direction with the light substrates 26 and 28 also oriented in a vertical direction. The support platform 20 may alternatively rotate in multiple possible directions within the radiation chamber in varying degrees from between 0° to 360°. Support platform 20 may also oscillate back and forth, or side to side along the same plane. As a further alternative, one or more of the light sources may also move in a coordinated manner with the movement of the support platform. Such oscillation or rotation would enable the majority of the photo sensitizer and fluid contained within the container 22 to be exposed to the light emitted from each of the discrete radiation sources (e.g. discrete sources 18 and 30), by continually replacing the exposed fluid at the light-fluid interface with fluid from other parts of the bag not yet exposed to the light. Such mixing continually brings to the surface new fluid to be exposed to light. The movement of both the support platform 20 and/or the radiation sources 14 and 16 may be controlled by control unit 24. The control unit 24 may also control the rate of light emission.

In a preferred embodiment each discrete light source 18 and 30 emits a peak wavelength of light to irradiate the fluid contained in bag 22. The peak wavelength of light emitted by each discrete light source is selected to provide irradiation of a sufficient intensity to activate both the photo sensitizer in a pathogen inactivation process as well as to provide sufficient penetration of light into the particular fluid being irradiated, without causing significant damage to the blood or blood components being irradiated. The preferred photo sensitizer is riboflavin. To irradiate a fluid containing red blood cells and riboflavin, it is preferred that each discrete light source 18 and 30 be selected to emit light at a peak wavelength of about 302 nm. Alternatively, 470 nm light might be used. The 470 nm of light is close to the optimal wavelength of light to both photolyse riboflavin, and also to enable significant penetration of the fluid containing red blood cells by the light.

If desired, the light sources 18 and 30 may be light emitting diodes and might be pulsed. Pulsing the lights may be advantageous because the intensity of light produced by the light sources may be increased dramatically if the lights are allowed to be turned off and rested between light pulses. Pulsing the light at a high intensity also allows for greater depth of light penetration into the fluid being irradiated, thus allowing a thicker layer of fluid to be irradiated with each light pulse.

The light sources 18, as shown in FIG. 2, may be fluorescent or incandescent tubes, which stretch the length of the irradiation chamber, or may be a single light source which extends the length and width of the entire chamber (not shown). LEDs may also be used in this embodiment. As shown in FIG. 2, the support platform 20 may be located within and/or forming part of a drawer 34. The support platform 20 may contain gaps 36 or holes or spaces within the platform 20 to allow radiation to penetrate through the gaps directly into the container 22 containing fluid to be irradiated.

A cooling system may also optionally be included. Air cooling using at least one fan 38 may be preferred but it is understood that other well-known systems can also be used. Although not shown in FIG. 2, the apparatus may also include temperature sensors and other cooling mechanisms where necessary to keep the temperature below temperatures at which desired proteins and blood components in the fluid being irradiated are damaged. Preferably, the temperature is kept between about 0° C. and about 45° C., more preferably between about 4° C. and about 37° C., and most preferably about 28° C.

The present invention includes a removable radiometer 40 that has the general shape of a blood bag 22. When placed on the support platform 20 and electrically connected to the controller 24, the radiometer 40 detects the intensity of incident light, preferably ultraviolet light, thereby allowing for calibration of the apparatus 10. Once calibrated, the controller 24 will be able to adjust exposure time and light intensity to deliver a desired dose of radiation to a blood bag and its contents. The support platform or platen 20 carries the radiometer 40 backwards and forwards parallel to the ultraviolet florescent light sources 18. The stroke distance allows the sensing apparatus (described below) of the radiometer to “view” the light sources 18, 30. Each of the light sources 18, 30 has an associated photo sensor (not shown) in electrical communication with the controller 24. During calibration, the controller 24 correlates the signals from the photo sensors to the output of the radiometer 40. When the radiometer 40 has been removed and replaced with a blood bag, the controller 24 will control the dose of radiation received by the blood bag based on the calibrated signals received from the photo sensors.

The radiometer 40 should produce accurate, stable measurements of the light intensity produced by the illuminator. Accurate, consistent measurements by the radiometer over an extended period of time allows for increased use of the illuminator between re-calibrations of the radiometer or extended maintenance of the illuminator. It is believed that the relative stability of the radiometer is strongly correlated to the optical characteristics of a reflecting surface in the radiometer. It is desirable, therefore, to provide an optical surface with stable optical characteristics over extended periods of time. The present invention provides a radiometer having such an optically stable surface and a method for producing the optical surface.

The radiometer 40 comprises at least one elongated, cylindrical optical chamber. If light is supplied solely from one side, for example, from the upper lamps 18, a first or upward-opening optical chamber 46 may be provided, oriented generally perpendicularly to the tubes 18 and to a reciprocating movement of the platen 20. An upper surface 48 of the radiometer 40 has a slot 50 parallel to the elongated axis of the optical chamber 46, which allows light from the lamps 18 to enter the optical chamber. The edges 52 of the slot 50 are preferably chamfered to allow light from most of the length of the lamps to be received in the chamber 46. Reciprocating movement of the platen 20 brings additional lengths of the lamps at each end into the view of the chamber 46. Thus, the radiation received through the slot 50 approximates the radiation received by a blood sample and sample bag along a line at the position of the slot. Because the chamber “averages” the non-uniform light field emitted by the lamps 18, the exposure on this line can be used to calculate the total exposure dose received by the sample.

An inner surface 54 of the optical chamber 46 is optically rough, allowing light received in the chamber to reflect within the chamber in such a way that the light field becomes averaged at any point within the chamber. A single photo sensor 56, mounted in the inner surface 54 perpendicularly from the slot 50, can sense an intensity representative of the radiation being received along the entire length of the slot. Preferably, the photo sensor 50 is recessed away from the inner surface 54, to reduce the likelihood of a beam of light or radiation from the lamps 18 falling directly on the photo sensor 56 without at least one reflection from the inner surface 54 of the chamber. Multiple photo sensors may also be used.

In an embodiment having a lower bank of lamps 30, the radiometer 40 preferably has a second downward-opening optical chamber 58. The second optical chamber 58 is oriented parallel to the first optical chamber 46 and also comprises a slot 60 in a lower surface 62 of the radiometer 40, the slot 60 being parallel to the elongated axis of the second optical chamber 58, but oriented in an opposite direction from the slot 50 in the first chamber 46, which allows light from the lower lamps 30 to enter the second optical chamber 58. The edges 64 of the slot 60 are preferably chamfered, as explained above, and reciprocating movement of the platen 20 brings additional lengths of the lower lamps at each end into the view of the lower chamber 58. An inner surface 66 of the optical chamber 58 is optically rough. As explained above, a single photo sensor 68, mounted in the inner surface 66 perpendicularly from the slot 60, is recessed away from the inner surface 66. Although a single photo sensor in each optical chamber is preferred, a plurality of photo sensors could also be used.

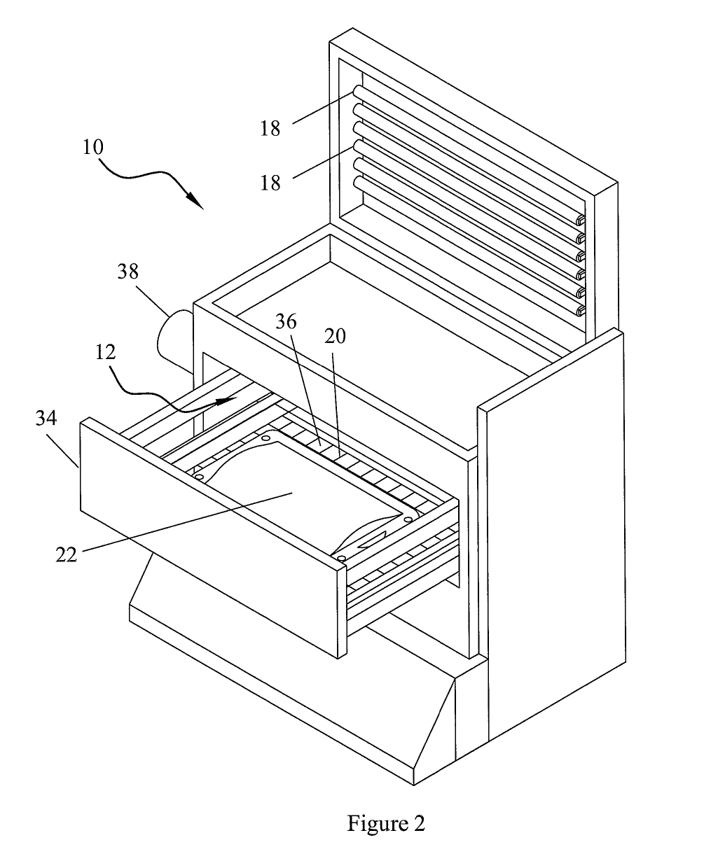

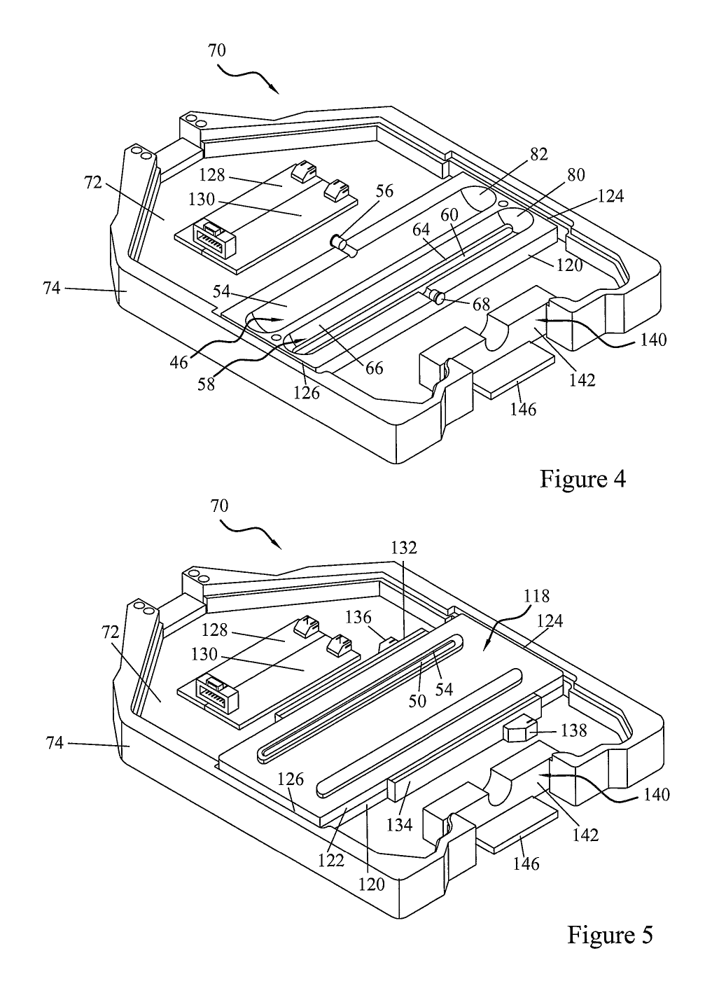

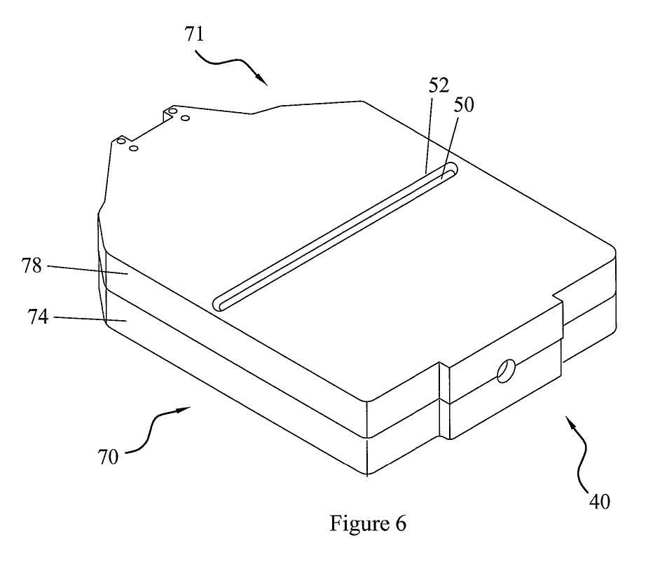

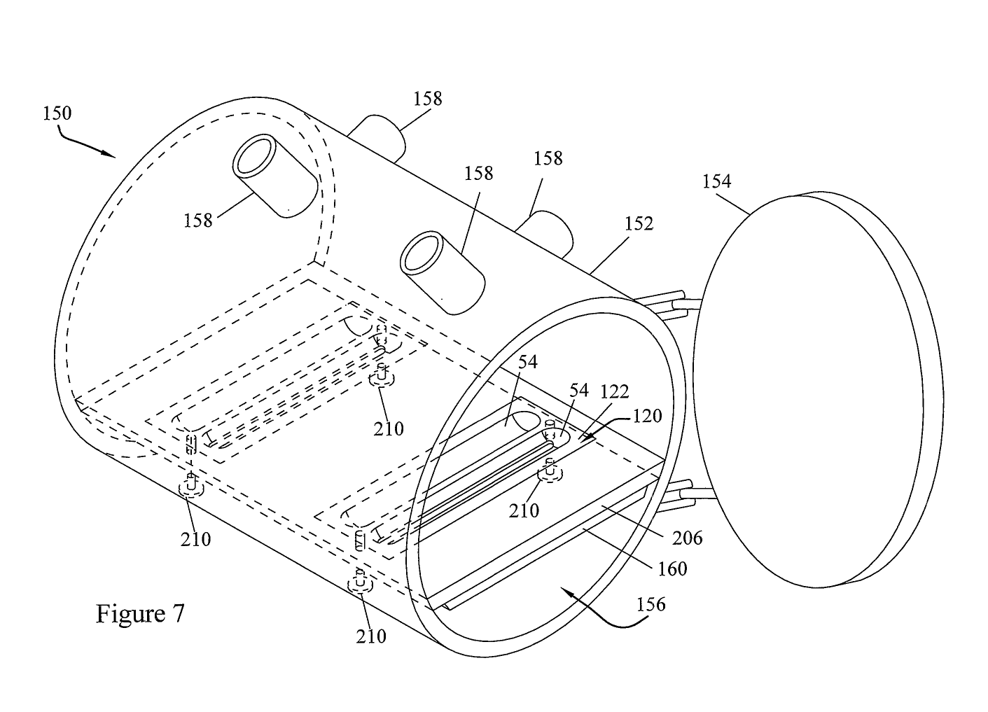

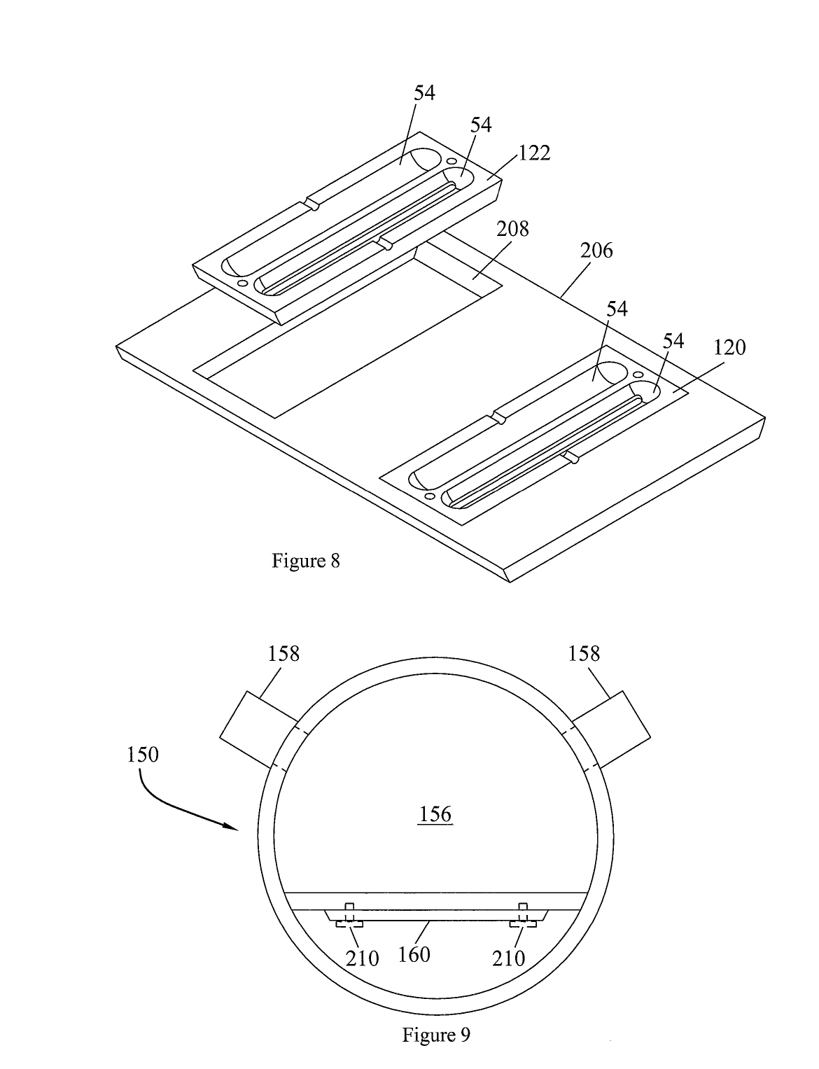

The radiometer 40 comprises an upper shell 71 and a lower shell 70. The lower shell 70, as shown in FIG. 4 and FIG. 5, has a bottom surface 72 and a peripheral wall 74, the peripheral wall having the general shape of a blood bag of a type that might be used in the illuminator 10. The upper shell 71 has a top surface 48 and a mating peripheral wall 78 (see FIG. 3), adapted to fit against the peripheral wall 74 of the lower shell 70. As explained above, first and second optical chambers 46, 58 are provided. These chambers 46, 58 comprise mating half-cylinders 80, 82 in the lower shell 70 and upper half-cylinders in the upper shell 71. In the embodiment shown in FIG. 4 and FIG. 5, the chambers 46, 58 are within an inner box 118 having a lower part 120 and an upper part 122. Heat sinks 132, 134 cover the photo diodes 56, 68 on the outside of the box 118. Thermistors 136, 138, in thermal contact with the heat sinks 132, 134 respond to the temperature of the heat sinks, which is also representative of the temperature of the photo diodes 56, 68. The output of the photodiodes is a function not only of the incident illumination, but also of the temperature of the photodiode. Thus, as the temperature of the photodiode increases, the output current of the photodiode will also rise, even if the illuminating radiation is constant. In order to provide an accurate measure of the illumination (as well as an accurate dose of radiation by the illuminator), the control circuit 24 compensates both for the temperature of the photodiodes in the radiometer during calibration and for the temperature of the photodiodes in the illuminator during viral inactivation. Electrical connecting wires (not shown) may pass through gaps 124, 126 between the shells 70, 71 and the box 118, providing electrical connections between the photo diodes 56, 68, amplifier circuits 128, 130, thermistors 136, 138 and the control unit 24. The wires pass as a cable through a block 140 (FIG. 3), comprised of two mating halves 142, 144, the lower half 142 of which is shown in FIG. 4 and FIG. 5. Spring plates 146, 148 may be provided adjacent the block on both the lower shell 70 and the upper shell 71, which may be engaged by a clamp (not shown) that holds a blood bag in position on the illuminator.

Each of the photo sensors 56, 68 is electrically coupled to transimpedance amplifiers 128, 130 respectively. The amplifiers 128, 130 are further electrically connected through a communications cable to the control unit 24. Male and female plugs (not shown) may be provided so that the radiometer may be selectively coupled to the control unit 24 for calibrating the apparatus, and then removed for ordinary operation.

As mentioned above, it is important to have an inner surface 54 on both the lower part 120 and the upper part 122 of the inner box 118 that is both optically rough and optically stable. Such a surface may be produced on an aluminum substrate by forming the lower and upper parts 120, 122 of a suitable aluminum with a minimum of other elements, such as 1100 aluminum, bead blasting at least the inner surface 54 and polishing the inner surface 54. Aluminum is inherently specular in its reflectivity. It is known to those skilled in the art, that if the surface roughness is on the order of the magnitude of the wavelengths of light reflecting off the surface, then the reflection will be diffuse in nature. It is impracticable using current methods of mass production machining operations to produce a diffusely reflective optical surface in this manner for this application. It can be demonstrated using integrating sphere theory that photons, upon entering the cavity, are largely randomized (i.e. spatial information associated with the photon has been lost) after three reflections within the cavity. The practical consequence of this is that a surface roughness that is much greater than wavelengths being reflected can be used to approximate diffuse reflectivity provided that care is taken with the design of the integrating cavity to ensure that “first strike, second strike, and third strike” light is prevented from reaching the detector. A surface roughness that is produced by using glass bead media of 60 to 100 grit is easily manufactured and sufficient for this application to approximate diffuse reflectivity. First, second, and third strike light is prevented from reaching the detector by use of a baffle that is located above the detector and additionally, bridges across the aperture, and by incorporating a setback of the detector away from (that is, behind) the optical surface of the cavity. The inner surface 54 may be further enhanced by plasma coating the inner surface in an oxygen ion atmosphere, thereby producing a layer of aluminum oxide (Al₂O₃), or artificial sapphire on the inner surface. It is believed that the coating should preferably be between 200 nm and 600 nm average thickness.

To achieve an appropriate surface, the reflective surfaces 54 and the test coupons were bead blasted with 60 to 100 grit glass beads, using new media, at 25 psi to produce a texture that is an approximation of a diffuse reflective surface.

Bead blasting additionally work hardens the optical surface by the introduction of dislocations to the aluminum substrate crystal structure. The dislocations produce compressive forces that are localized within the region of the optical surface. The compressive forces that bead blasting introduces has an effect that further improves stability of the optical surface compared to those optical surfaces that have not been bead-blasted. It is hypothesized that the compressive forces improve stability by precluding the introduction and subsequent growth of micro fractures that can provide sites and opportunities for corrosion to occur. The resulting surfaces were then electro polished by Able Electropolish of Chicago, Ill. The electro polishing should create a minor-like surface on the micro-features of the reflective 54 surface, but should not remove all of the surface variation caused by the bead blasting. The electro polishing should also remove mechanically disturbed metal that has been produced by prior machining operations and any contaminants, such as fragments of the glass beads. The electro polished surface is then chemically cleaned and ready for plasma treatment.

Plasma treatment, including plasma cleaning and oxidation, of the bead blasted, polished and chemically cleaned surface 54 is preferably performed in a plasma treatment chamber 150. A suitable system is the 3005/BJR laboratory system from Plasmatech of Erlanger, Kentucky. The plasma chamber 150 comprises a sealable cylinder 152 with a door 154. The cylinder 152 and door 154 form a sealed area 156 that can maintain a vacuum or low-air pressure state. Argon and Oxygen flow into the chamber 152 through microwave source port 158. RF substrate biasing is introduced through the RF feedthrough 159. The microwave sources 158 convert oxygen and argon into oxygen atoms and oxygen and argon ions, which form a plasma within the chamber. A plate 160 supports objects that may be bombarded by the oxygen ions. RF biasing power, applied to the substrate, creates a negative DC voltage that accelerates ions toward the plate 160. As the oxygen atoms and ions strike an aluminum surface, such as the reflecting surface 54, aluminum oxide (Al₂O₃) is formed. The following table contains stability data for n=2 cavities that have been plasma treated, as described above.

| Plasma Process Parameters | Pre-treatment | Batch |
|---|---|---|
| Microwave (watts) | 800 | 1000 |
| Low Freq (watts) | 200 | 100 |
| Time (seconds) | 600 | 3600 |
| Pressure (Pa) | 60-80 | 100-120 |
| O2 Flow (sccm) | 800-1000 | 600 |

| | |
|---|---|
| Measured | % Change in |
| Target Value | Target Value |

| | | | |
|---|---|---|---|
| Side 1 | Side 2 | Side 1 | Side 2 |

1. As Calibrated

| | | | | |
|---|---|---|---|---|
| Cavity Set 1 | 2024 | 2024 | 0.00% | 0.00% |
| Cavity Set 2 | 2024 | 2024 | 0.00% | 0.00% |

2. After 72 hours of 38° C.
and 85% RH

| | | | | |
|---|---|---|---|---|
| Cavity Set 1 | 2029 | 2027 | −0.25% | −0.15% |
| Cavity Set 2 | 2008 | 1993 | 0.79% | 1.53% |

3. After 72 hours of UV Desorption

| | | | | |
|---|---|---|---|---|
| Cavity Set 1 | 2023 | 2028 | 0.05% | −0.20% |
| Cavity Set 2 | 2016 | 2007 | 0.40% | 0.84% |

The two sets of cavities that were used for the plasma treatment for the above table had a machined surface roughness prior to bead-blasting of approximately 2000 u-inches. This level of surface roughness produces substantial amounts of mechanically disturbed metal on the optical surface that cannot be entirely removed by electro polishing. The specification for the machined surface roughness is preferred to be 32 u-inches.

Suitable conditions for plasma treatment have been determined using planar test coupons, which were placed on the plate 160, coated and tested for optical stability as described below. Test conditions for each plasma treatment, PLAT (or plasma treatment 1) through PLA6 are set forth in the following Table 1:

TABLE 1

| | Microwave | Low | | | Oxygen |
|---|---|---|---|---|---|
| | Power | Frequency | Time | Pressure | Flow |
| | (Watts) | Bias (Watts) | (Seconds) | (Pa) | (sccm) |
| Pre-Treat | 800 | 200 | 600 | 30 | 800-1000 |
| PLA1 | 600 | 200 | 3600 | 50-60 | 600 |
| PLA2 | 600 | 200 | 7200 | 50-60 | 600 |
| PLA3 | 800 | 200 | 7200 | 50-60 | 600 |
| PLA4 | 1000 | 100 | 3600 | 50-60 | 600 |
| PLA5 | 1000 | 100 | 7200 | 50-60 | 600 |
| PLA6 | 1000 | 100 | 1800 | 50-60 | 600 |

After bead blasting, electro polishing and plasma treatment (except for the control, as described below), the test coupons were exposed to elevated temperatures and high humidity, simulating adverse conditions that might be encountered in use. The optical characteristics of the coupons were measured over a period of days. The coupons were generally maintained at about 38 degrees Celsius and 85% relative humidity.

Control coupons were bead-blasted and electro polished, but not plasma treated. The changes in reflectivity due to humidity exposure were most pronounced in the region from 250 nm to 320 nm. Aluminum hydroxide Al(OH)₃ and aluminum oxide hydroxide AlO(OH) absorb UV more strongly in this region. The coupons were subsequently exposed to a high intensity UV source for three days, that is, “UV Cooked”. High intensity UV breaks the hydroxide bonds producing amorphous aluminum oxide Al₂O₃. After several days, the percentage reflectivity (% R) for the control coupons, after exposure to heat and humidity and subsequent UV “cooking”, was found to be:

| | % R Control | | | | | |
|---|---|---|---|---|---|---|
| | 0 Days | 3 Days | 7 Days | 10 Days | 14 Days | UV Cooked |
| Average | 80.52% | 77.91% | 76.89% | 76.02% | 74.86% | 77.37% |
| % R | | | | | | |

PLA6 had the highest average percentage reflectivity of all the plasma treated coupons. A high percentage reflectivity optical surface in the integrating cavity is optimal for producing a signal that is representative of the average irradiance of the non-uniform light field that is present at the aperture. PLA6 demonstrated relatively good stability for ten days of humidity exposure. However, it had a significant change in reflectivity after fourteen days exposure. The observed change in reflectivity appears to be largely reversible upon exposure to high intensity UV. PLA6, PLA4, and PL5 were processed using the same power settings for 30, 60, and 120 minutes respectively. It was estimated that the PLA6 Al₂O₃ thin film was approximately 200 nm thick based upon cross-section FESEM measurements of the PLA4 thin film at 400 nm.

The estimated 200 nm thickness of the PLA6 layer was thought to be responsible for the higher percentage reflectivity and its reduced optical stability compared to PLA4.

| | % R PLA6 | | | | | |
|---|---|---|---|---|---|---|
| | 0 Days | 3 Days | 7 Days | 10 Days | 14 Days | UV Cooked |
| Average | 78.80% | 78.73% | 78.38% | 77.88% | 76.96% | 78.26% |
| % R: | | | | | | |

PLA3 had a relatively high percentage reflectivity with an average thin film thickness of 970 nm. However, its optical stability was poor and its change in reflectivity upon exposure to humidity appeared to be largely irreversible upon subsequent exposure to UV. This is suggestive of the formation of a lower density and a more amorphous Al₂O₃ thin film that could occur as a result of the lower power setting that was used compared to PLA6.

| | % R PLA3 | | | | | |
|---|---|---|---|---|---|---|
| | 0 Days | 3 Days | 7 Days | 10 Days | 14 Days | UV Cooked |
| Average | 77.25% | 75.17% | 74.40% | 74.53% | 74.13% | 74.48% |
| % R: | | | | | | |

PLA1 had a performance (% R, Optical Stability, UV Reversibility) that was intermediate between the best (PLA6) and the worst (PLA3) of the test samples. PLA1 had an average thin film thickness of 530 nm. PLA1 and PLA2 were processed using the same power settings for 60 and 120 minutes respectively.

| | % R PLA1 | | | | | |
|---|---|---|---|---|---|---|
| | 0 Days | 3 Days | 7 Days | 10 Days | 14 Days | UV Cooked |
| Average | 74.52% | 72.68% | 72.81% | 72.36% | 72.34% | 73.78% |
| % R: | | | | | | |

PLA4 had a relatively low percentage reflectivity with an average thin film thickness of 400 nm. However, its optical stability was the best of all samples evaluated. Of particular note with PLA4 was the absence of a pronounced curvature in the drop-off in reflectivity from 250 nm to 300 nm as is seen in the other samples. This indicates that little aluminum hydroxide develops from exposure to humidity. The observed change in reflectivity was effectively reversible upon exposure to high intensity UV suggesting the desorption of molecular water that was present on the optical surface.

| | % R PLA4 | | | | | |
|---|---|---|---|---|---|---|
| | 0 Days | 3 Days | 7 Days | 10 Days | 14 Days | UV Cooked |
| Average | 70.91% | 70.30% | 69.38% | 70.32% | 69.90% | 70.78% |
| % R: | | | | | | |

PLA2 had a relatively low percentage reflectivity with an estimated average thin film thickness of approximately 1000 nm. Its optical stability was intermediate between the best (PLA6) and the worst (PLA3) of the test samples and its change in reflectivity upon exposure to humidity appeared to be largely irreversible upon subsequent exposure to UV. It is hypothesized that tensile strains in the Al₂O₃ film introduced cracks due to the thickness of the film.

| | % R PLA2 | | | | | |
|---|---|---|---|---|---|---|
| | 0 Days | 3 Days | 7 Days | 10 Days | 14 Days | UV Cooked |
| Average | 69.43% | 68.32% | 67.27% | 68.26% | 68.15% | 68.22% |
| % R: | | | | | | |

PLA5 had the lowest percentage reflectivity with an average thin film thickness of 1100 nm. Its optical stability was relatively low despite the thickness of the thin film and its change in reflectivity upon exposure to humidity appears to be relatively irreversible upon subsequent exposure to UV. It is hypothesized that tensile strains in the Al₂O₃ film introduced cracks due to the thickness of the film.

| | % R PLA5 | | | | | |
|---|---|---|---|---|---|---|
| | 0 Days | 3 Days | 7 Days | 10 Days | 14 Days | UV Cooked |
| Average | 69.38% | 67.97% | 67.19% | 66.35% | 66.55% | 67.51% |
| % R: | | | | | | |

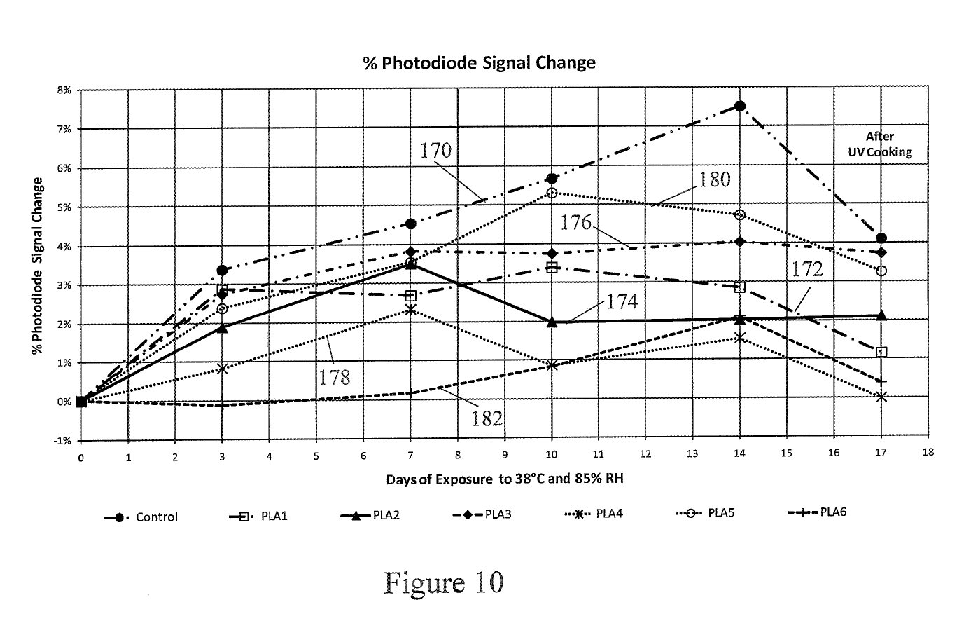

FIG. 10 plots the percent change in photodiode signal for plasma treated coupons exposed to 38° C. and 85% relative humidity for fourteen days. The coupons were subsequently exposed to a high intensity UV source for three days. The uncoated control coupon is shown at line 170. The PLA1 coupon is shown at line 172; the PLA2 coupon, at line 174; the PLA3 coupon, at line 176; the PLA4 coupon, at line 178; the PLA5 coupon, at line 180; and the PLA6 coupon, at line 182. All plasma treated coupons are more optically stable than untreated coupons. PLA4 and PLA6 plasma treated coupons are the most stable.

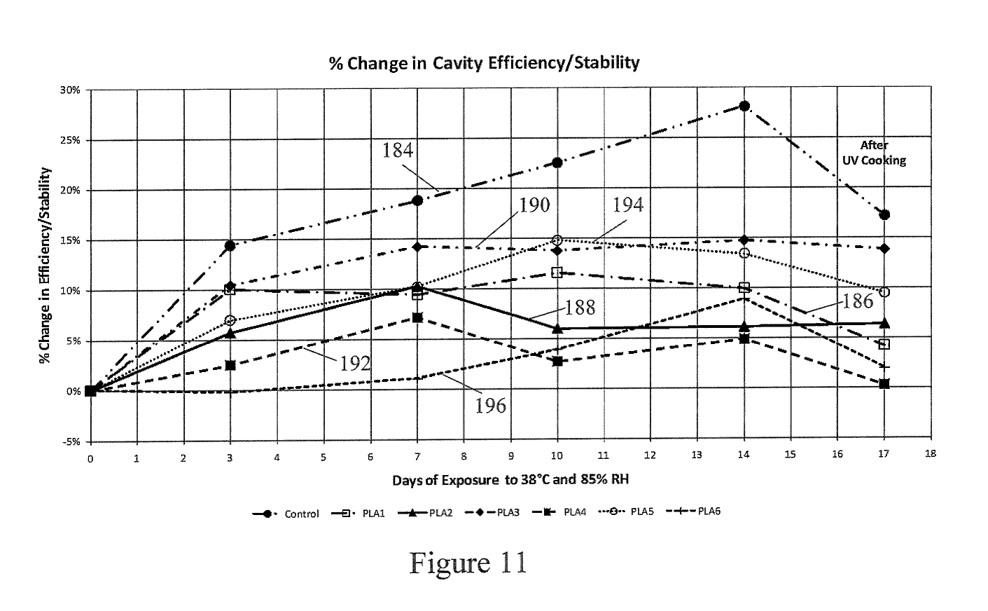

FIG. 11 is a plot of the percent change in cavity efficiency/stability for plasma treated coupons exposed to 38° C. and 85% relative humidity for fourteen days. The coupons were subsequently exposed to a high intensity UV source for three days. Efficiency is a measure of an integrating cavity's ability to remove spatial information from light that has passed through the cavity aperture. Spatial information is removed by “randomizing” the photons by means of multiple reflections within the cavity. Efficiency is therefore a function of the reflectivity of the optical surface of the cavity. The untreated control coupon is shown at line 184. The PLA1 coupon is shown at line 186; the PLA2 coupon, at line 188; the PLA3 coupon, at line 190; the PLA4 coupon, at line 192; the PLA5 coupon, at line 194; and the PLA6 coupon, at line 196. Of particular note, highly efficient integrating cavities are also intrinsically unstable. The performance of the integrating cavity radiometer is therefore a tradeoff between efficiency and stability. All plasma treated coupons are more optically stable than untreated coupons. PLA4 and PLA6 plasma treated coupons are the most stable.

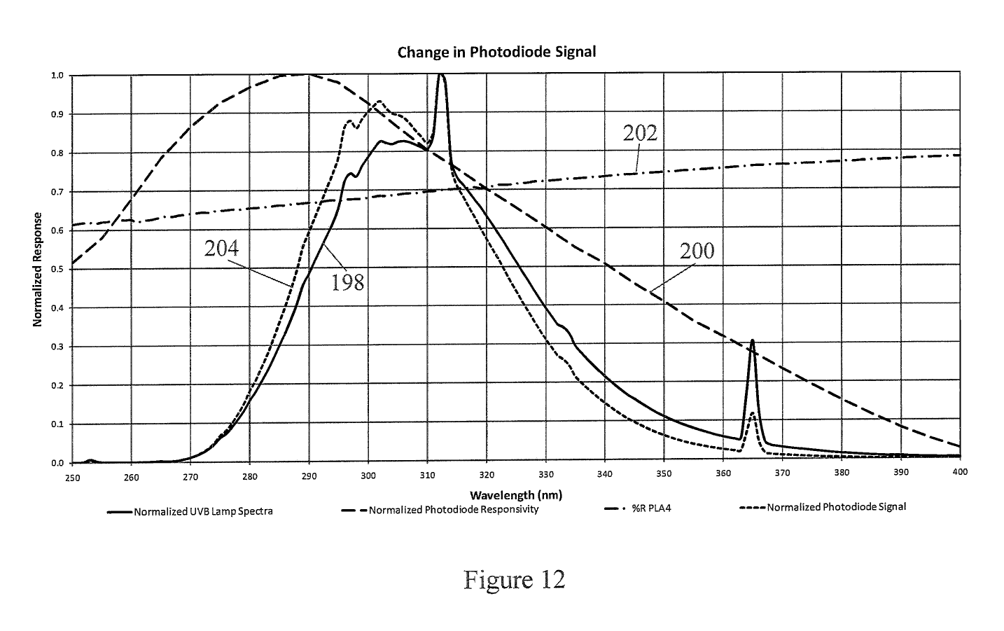

The change in spectral reflectivity of the coupons due to humidity can be correlated, as shown in FIG. 12, to a change observed in the radiometer from the photodiode signal by convolving. Convolution is a mathematical operation on two functions f and g, producing a third function that is typically viewed as a modified version of one of the original functions. FIG. 12 shows the normalized UVB lamp spectra 198 measured directly from the lamps. The output of the lamps peaks near 308 nm and show a prominent energy spike near 365 nm. It is desirable for the output signal of the radiometer to be closely correlated to the spectral output of the lamp, so that an appropriate dosage can be calculated and delivered to a fluid (e.g., blood) in the fluid container 22 by the illuminator apparatus 10. As shown in FIG. 12, the photodiode responsivity 200 is a relatively smooth curve, with maximum response at about 290 nm. A percentage reflectivity 202 for a reflective surface, in this example the percentage reflectivity for PLA4, is also shown. By mathematical convolution of the photodiode signal (not shown) with the photodiode responsivity 200 and the percentage reflectivity 202, a normalized photodiode signal 204 may be obtained that closely follows the UVB lamp spectral output 198. A photodiode signal current can be obtained by integration of the area under the normalized photodiode signal 204.

Thus, a suitable reflective surface for a radiometer for calibrating a biologic fluid illuminator may comprise a bead-blasted aluminum surface, electro polished to create a mirror-like surface on the micro-features of the reflective 54 surface, but not to remove all of the surface variation caused by the bead blasting. The electro polishing should also remove any mechanically disturbed metal fragments or sharp edges and any contaminants, such as fragments of the glass beads. The reflective surface may further comprise an aluminum oxide layer. Preferably the aluminum oxide layer is between about 200 nm and 600 nm thick. The aluminum oxide layer may be produced by oxygen plasma treatment of an aluminum substrate.

The test information was obtained from sample coupons having planar surfaces for exposure to the ionized oxygen in the ion chamber 150. In the preferred embodiment, the reflective surfaces 54 are curved recesses, as shown in FIG. 4, for example. In order to obtain the desired coating thickness, length of treatment of the lower and upper parts 120, 122 in the ion chamber 150 may be required. For curved surfaces, it is believed that a coating thickness of 400 nm to 600 nm may provide the best stability. To calibrate a pathogen inactivation apparatus, the radiometer 40 is substituted for a blood bag, and occupies the same location in the apparatus and has the same general shape as a blood bag containing blood or blood components. The radiometer would be electrically connected to the control unit 24 and exposed to radiation from the lamps 18, 30 for a selected period of time. Preferably, the platen 20 would also be agitated it the same manner as when a blood sample would be treated in the apparatus. The output of the radiometer provides a benchmark to the control unit 24 of exposure intensity per unit time, from which a desired dose of radiation can be calculated. After calibration of the apparatus, units of blood or blood components in appropriate translucent or transparent bags can be placed in the pathogen inactivation apparatus and exposed to controlled quantities of radiation.

In view of the foregoing, it is believed that the plasma treatment for the curved surface of the radiometer should preferably comprise the steps shown in the following table:

| Process | Pre-Treat - Step 1 | Pre-Treat - Step 2 | Process Batch |
|---|---|---|---|
| Microwave | 800 | 800 | 1000 |
| (watts) | | | |
| Low Freq (watts) | 200 | 200 | 100 |
| Time (seconds) | 300 | 600 | 3600 |
| Pressure (Pa) | 85-105 | 85-105 | 140-160 |
| O2 Flow (sccm) | | 800-1000 | 600 |
| Ar Flow (sccm) | 800-1000 | | |

Pre-treatment step 1 comprises plasma cleaning of residual contamination using argon plasma. Pre-treatment step 2 comprises aluminum oxidation and removing of aluminum hydroxide. The batch process provides deep aluminum oxidation, which grows a thick aluminum oxide layer. It is believed that the argon pre-treatment is non-reactive and will “blast” any non-hydrocarbon residues from the surface. The oxygen pre-treatment will remove aluminum hydroxide and “burn off” any hydrocarbons from the surface. The pretreatment steps will ensure a clean aluminum substrate to produce a high quality Al₂O₃ layer. It is believed that the oxygen pressure should be increased to improve penetration of atomic and ionized oxygen into the cavity, producing a more uniform surface thickness of the Al₂O₃ layer.

It will be apparent to those skilled in the art that various modifications and variations can be made to the structure and methodology of the present invention without departing from the scope or spirit of the invention. Rather, the invention is intended to cover modifications and variations provided they come within the scope of the following claims and their equivalents.

What is claimed is:

1. An irradiation apparatus for inactivating pathogens or white blood cells in a fluid containing blood components comprising:
at least one radiation emitting source emitting radiation; and
a control unit for controlling the radiation emitting source,
a radiometer in electrical communication with said control unit, said radiometer comprising a first optical chamber having an aperture for receiving at least some of said radiation and a photo sensor responsive to said received radiation in said optical chamber, said optical chamber having a bead-blasted aluminum reflective surface.

2. The irradiation apparatus of claim 1 wherein said reflective surface is polished.

3. The irradiation apparatus of claim 2 wherein said reflective surface further comprises a layer of aluminum oxide.

4. The irradiation chamber of claim 3 wherein said layer of aluminum oxide is at least about 200 nm in thickness.

5. The irradiation chamber of claim 4 wherein said layer of aluminum oxide is at most about 600 nm in thickness.

6. The irradiation chamber of claim 5 wherein said layer of aluminum oxide is formed on said aluminum reflective surface by consecutive plasma treatments including plasma cleaning and oxidation.

7. A method of making a radiometer comprising
forming a concave reflective surface in an aluminum part,
bead-blasting said surface,
polishing said surface,
securing said part to the RF electrode,
mounting said part and said cradle in a plasma pretreatment apparatus, and having consecutive steps of plasma cleaning and plasma oxidation to form a layer of aluminum oxide.

8. The method of claim 7 wherein said plasma oxidation forms said layer of aluminum oxide to a thickness of at least 200 nm.

9. The method of claim 8 wherein said plasma oxidation forms said layer of aluminum oxide to a thickness of not more than 600 nm.

10. A radiometer for measuring ambient light, said radiometer comprising a first optical chamber having an aperture for receiving at least some of said radiation and a photo sensor responsive to said received radiation in said optical chamber, said optical chamber having a bead-blasted aluminum reflective surface.

11. The radiometer of claim 10 wherein said reflective surface is polished.

12. The radiometer of claim 11 wherein said reflective surface further comprises a layer of aluminum oxide.

13. The radiometer of claim 12 wherein said layer of aluminum oxide is at least about 200 nm in thickness.

14. The radiometer of claim 13 wherein said layer of aluminum oxide is at most about 600 nm in thickness.

15. The radiometer of claim 14 wherein said layer of aluminum oxide is formed on said aluminum reflective surface by plasma treatment.

16. An apparatus for providing a diffuse reflective surface with stable optical properties, the apparatus comprising:
a polished, bead-blasted aluminum reflective surface, said surface having a layer of aluminum oxide,
said layer of aluminum oxide being at least about 200 nm in thickness and at most about 600 nm in thickness.

17. A method of making a diffuse reflective surface with stable optical properties, said method comprising
forming a reflective surface in an aluminum part,
bead-blasting said surface,
polishing said surface,
mounting said part in a plasma pre-treatment apparatus, and having consecutive steps of plasma cleaning and plasma oxidation to form a layer of aluminum oxide to a thickness of at least 200 nm.

18. The method of claim 17 wherein said ions and atoms form said layer to a thickness of not more than 600 nm.
